SEQUENCE LISTING (8 sequences)

SEQ ID NO: 1 - DNA, 22 bp, Artificial Sequence
  Synthesized
tcgtcgttctgccatcgtcgtt

SEQ ID NO: 2 - DNA, 21 bp, Artificial Sequence
  Synthesized
tcgtcgttcggccgatcgtcc

SEQ ID NO: 3 - DNA, 14 bp, Artificial Sequence
  Synthesized
ggggtcgtctgcgg

SEQ ID NO: 4 - DNA, 16 bp, Artificial Sequence
  Synthesized
gatcgtccgtcggggg

SEQ ID NO: 5 - DNA, 21 bp, Artificial Sequence
  Synthesized
tcgttcgtctgcttgttcgtc

SEQ ID NO: 6 - DNA, 24 bp, Artificial Sequence
  Synthesized
ccggggtcgcagctgagcccacgg

SEQ ID NO: 7 - DNA, 19 bp, Artificial Sequence
  Synthesized
ggaacagttcgtccatggc

SEQ ID NO: 8 - DNA, 19 bp, Artificial Sequence
  Synthesized
ggaacagttgctccatggc

(19) United States
(12) Patent Application Publication
(10) Pub. No.: US 2011/0280934 A1
(43) Pub. Date: Nov. 17, 2011

(54) Increased Expression of Specific Antigens

(76) Inventors: Asa Karlsson, Sollentuna (SE); Lisa Bandholtz, Stockholm (SE); Nikolai Kouznetsov, Jarfalla (SE); Oliver Von Stein, Upplands Vasby (SE); Petra Von Stein, Upplands Vasby (SE); Arezou Zargari, Solna (SE)

(21) Appl. No.: 13/127,288

(22) PCT Filed: Nov. 3, 2009
(86) PCT No.: PCT/SE2009/051244

§ 371 Date: Jul. 25, 2011

Related U.S. Application Data
(60) Provisional application No. 61/111,288, filed on Nov. 4, 2008.
(60) Provisional application No. 61/111,292, filed on Nov. 4, 2008.
(60) Provisional application No. 61/111,293, filed on Nov. 4, 2008.

(30) Foreign Application Priority Data
Nov. 4, 2008 (SE) 0802335-0
Nov. 4, 2008 (SE) 0802336-8
Nov. 4, 2008 (SE) 0802337-6

(51) Int. Cl. A61K 9/127; A61K 31/7125; A61P 37/04; A61P 35/00; A61P 25/00; C07H 21/04; A61K 39/395

(52) U.S. Cl. 424/450; 536/23.1; 424/184.1; 424/277.1; 424/173.1

(57) ABSTRACT

The response of immunotherapy in the treatment of cancer is enhanced by use of an oligonucleotide in a dose effective to induce at least one of endogenous production of cytokines, and the regulation of the expression of one or more of the cell surface antigens (FIG. 1).

### TECHNICAL FIELD

The present inventions concern the field of immunotherapy and in particular the increased expression of CD20, CD23, CD69 and CD80 and makes available methods and compounds for this use.

### BACKGROUND

The use of cell surface antigens as therapeutic targets is a growing area of interest. Immunotherapy is used in the treatment or alleviation of many immunological diseases or conditions, such as cancer, inflammatory diseases such as asthma, allergy etc, including autoimmune disorders such as multiple sclerosis (MS). Monoclonal antibodies are offering substantial advantages in terms of potency, reproducibility and freedom from contaminants. It is in the areas of transplantation, cancer treatment and autoimmune disease that recent discoveries in immunology are having new impact.

There are several options of using antibody-related therapies. The antibody can be used to bind to a specific target molecule on the cell surface to trigger cellular mechanisms such as apoptosis or activation pathways (immunotherapy), or simply bind to a target on the cell surface for delivery of an agent to the specific cell type, e.g. a cytostatic agent (immune-chemo therapy) which could be combined with e.g. irradiation therapy.

Antibody mediated immunotherapy has been used in bone marrow transplantations, where antibodies are used to remove donor T-cells and prevent graft-versus-host disease; in many types of different cancers such as leukemia and lymphoma; as well as for the treatment of some autoimmune diseases such as rheumatoid arthritis, vasculitis and MS. Examples of these antibodies include anti-CD52 (for depleting lymphocytes), anti-CD25 (specific for activated T-cells), anti-CD4 (for blocking the function of the critical T-helper cells) and anti-CD18 (which blocks the migration of leukocytes from the blood to sites of inflammation).

Several antibodies have recently been approved for the treatment of cancer. The anti-CD20 antibody rituximab, which is a genetically engineered chimeric murine/human monoclonal antibody directed against human CD20 (Rituxan® or MabThera®, from Genentech Inc., South San Francisco, Calif., U.S.) is used for the treatment of patients with relapsed or refractory low-grade or follicular, CD20 positive, B-cell non-Hodgkin's lymphoma. Rituximab works by recruiting the body's natural defences to attack and kill the B-cell to which it binds via the CD20 antigen. In vitro mechanism of action studies have demonstrated that rituximab binds human complement and lyses lymphoid B-cell lines through complement-dependent cytotoxicity (CDC) (Reff et al., 1994). Additionally, it has significant activity in assays for antibody-dependent cellular cytotoxicity (ADCC). In vivo preclinical studies have shown that rituximab depletes B-cells from the peripheral blood, lymph nodes, and bone marrow of cynomolgus monkeys, presumably through complement and cell-mediated processes (Reff et al., 1994). Analysis of chronic lymphocytic leukemia (CLL) patients shows that the density of CD20 on the surface of B-CLL cells is rather variable with some patient's B-cells expressing very low levels of the CD20 antigen. The typical treatment for B-cell malignancies, besides rituximab, is the administration of radiation therapy and chemotherapeutic agents. In the case of CLL, a combination of two or three chemotherapies is often used to destroy malignant cells. However, side effects are a limiting factor in this treatment.

Another monoclonal antibody used is alemtuzumab (Campath® or MabCampath®, an anti-CD52 from Ilex Pharmaceuticals) (Keating, et al., 2002) which was approved for the treatment of CLL in 2001. Bevacizumab (Avastin®, Genentech, Inc., South San Francisco, Calif.) is a humanized IgG1 mAb directed against vascular endothelial growth factor (VEGF) used in treatment of colorectal cancer, small cell lung cancer and breast cancer. Trastuzumab (Herceptin® from Roche) is a humanized IgG1 mAb that is effective against metastatic breast cancer tumours over-expressing the HER-2 target (Strome et al., 2007).

Molecular engineering has improved the prospects for antibody-based therapeutics, resulting in different constructs and the development of humanized or human antibodies that can be frequently administered.

Another widely used treatment for haematological malignancies is chemotherapy. Combination chemotherapy has some success in reaching partial or complete remissions. Unfortunately, the remissions obtained through chemotherapy are often not durable.

Currently, a number of other monoclonal antibodies are being investigated for MS, including some that are already in use in other conditions. These include ocrelizumab (Genetech/Hoffmann-La Roche), daclizumab (Biogen Idec, Inc.), alemtuzumab (Campath®, MabCampath®, Bayer Schering, BTG, Genzyme, Millenium) and rituximab (Rituxan®, MabThera®, Genentech, Hoffmann-La Roche, Biogen Idec Inc.) In 2004, the FDA approved the use of a monoclonal antibody (Natalizumab, Tysabri®, Biogen Idec Inc., Cambridge, Massacheusetts, USA, and Elan Pharmaceuticals, Inc., Dublin, Ireland) for the treatment of patients with relapsing forms of MS (FDA News PO4-107, Nov. 23, 2004). While generally well tolerated, natalizumab is occasionally associated with severe adverse effects.

Antibody therapy in general is costly, and there is a need for improvements inter alia with regards to efficacy.

Synthetic CpG oligonucleotides, CpG-ODNs, are a new class of immuno-modulatory agents that stimulate the immune system. Recent studies demonstrate that at least three classes of CpG-ODN sequences exist, each with different physical characteristics and biological effects. Preliminary studies in several animal models of cancer suggest that CpG-ODNs may have many uses in cancer immunotherapy. CpG-ODNs have the ability to induce tumour regression by activating innate immunity, and serving as potent vaccine adjuvants that elicit a specific, protective immune response. Early clinical trials indicate that CpG-ODNs can be administered safely to humans, and studies are ongoing to understand how these agents may play a role in cancer immunotherapy (Wooldridge, J E, et al. 2003). There are indications that the CpG motif alone is not accountable for the efficacy of the CpG-oligonucleotides. There are even indications that this motif is not necessary for the desired function, but that the backbone structure also is of importance for their immunomodulatory effects.

Immunotherapy of cancer has been explored for over a century, but it is only in the last decade that various antibody-based products have been introduced into the management of patients with diverse forms of cancer. At present, this is one of the most active areas of clinical research, with eight therapeutic products already approved in oncology. Antibodies against tumour-associated markers have been a part of medical practice in immunohistology and in vitro immunoassays for several decades, and are now becoming increasingly recognized as important biological agents for the detection and treatment of cancer (Strome et al., 2007).

CD20 is variably expressed on the surface of B-cells in CLL patients, with some patients' B-cells expressing very low levels of the CD20 antigen. CD20 (human B-lymphocyte restricted differentiation antigen), is a hydrophobic transmembrane protein with a molecular weight of approximately 35 kD located on pre-B and mature B lymphocytes. The antigen is also expressed on more than 90% of B-cells in non Hodgkin's lymphomas (NHL), but is not found on hematopoietic stem cells, pro B-cells, normal plasma cells or other normal tissues. CD20 regulates an early step(s) in the activation process for cell cycle initiation and differentiation, and possibly functions as a calcium ion channel. CD20 is not shed from the cell surface and does not internalize upon antibody binding. Free CD20 antigen is not found in the circulation (Pescovitz, 2006).

The expression of CD23 is highly up-regulated in normal activated follicular B-cells and has been found to be consistently present at higher levels in B-CLL cells. The CD23 leukocyte differentiation antigen is a 45 kD type II transmembrane glycoprotein expressed on several haematopoietic lineage cells, which function as a low affinity receptor for IgE (FcγRII) (Pathan et al., 2008). It is a member of the C-type lectin family and contains an α-helical coiled-coil stalk between the extracellular lectin binding domain and the transmembrane region. The stalk structure is believed to contribute to the oligomerization of membrane-bound CD23 to a trimer during binding to its ligand (for example, IgE). Upon proteolysis, the membrane bound CD23 gives rise to several soluble CD23 (sCD23) molecular weight species (37 kD, 29 kD and 16 kD). In addition to being involved in regulating the production of IgE, CD23 has also been speculated to promote survival of germinal center B-cells. Lumiliximab is a monoclonal chimeric anti-CD23 antibody (Biogen Idec, San Diego, USA) that harbours macaque variable regions and human constant regions (IgG1, K) and was originally developed to inhibit the production of IgE by activated human blood B-cells. It is currently undergoing two Phase II trials for use in B-CLL patients. In vitro studies have shown that lumiliximab induces caspase dependent apoptosis in B-CLL cells through the mitochondrial death pathway (Pathan et al., 2008). Thus, it seems to induce apoptosis of tumour cells through a mechanism different from rituximab.

The protein CD80 is a molecule found on activated B-cells, macrophages/monocytes and dendritic cells which provides a co-stimulatory signal necessary for T-cell activation and survival. It is also known as B7.1. Contact between B and T-cells can be mediated by antigen presentation, as well as antigen-independent cell interaction by adhesion molecules and co-stimulatory molecules (CD80, CD86, CD40). Its principal mode of action is by binding to CD28. Along with CD86, these molecules provide the necessary stimuli to prime T-cells against antigens presented by antigen-presenting cells. CD80 and CD86 also bind to CTLA-4, a cell surface molecule expressed on activated T-cells. Interactions between CD80 or CD86 with CTLA-4 decrease the response of T-cells.

The anti-CD80 monoclonal antibody galiximab (Biogen Idec) has been studied as a single-agent in previously treated follicular lymphoma (FL) and in combination with the anti-CD20 antibody rituximab against relapsed FL. In addition to B-cells, macrophages/monocytes and dendritic cells, the CD80 molecule is also found on the surface of activated macrophages, dendritic cells and cells from various subtypes of non Hodgkin's lymphoma (NHL). Galiximab's potential mechanisms of action include ADCC and possible immunomodulatory effects on host effector cells affecting the tumor microenvironment. Galiximab seems to induce apoptosis of tumor cells through mechanisms similar to rituximab. Thus, there is good reason to believe that an upregulation of CD80 surface expression enhance galiximab-induced apoptosis of cancer cells.

CD69 is a type II integral membrane protein with a single transmembrane domain belonging to the C-type lectin family of surface receptors with a molecular weight of 60 kDa. CD69 is a leukocyte receptor transiently induced after activation and is detected on NK-cells and small subsets of T and B-cells in peripheral lymphoid tissues from healthy subjects.

Multiple sclerosis (MS) is an autoimmune disease that affects the central nervous system, i.e. the brain and spinal cord. Inflammation destroys the myelin, leaving multiple areas of scar tissue (sclerosis) and the nerve impulses are slowed down or blocked. The inflammation occurs when the body's own immune cells attack the nervous system.

Therapy with antibodies (and other biological molecules) is limited by the ability of the patient's immune system to detect the new substance and neutralise it. Some cells express too little antigen to be targeted and cells in a large mass may escape attention altogether. One novel aspect of immuno-therapies is that they are based on the natural molecules which normally operate in concert to modulate and control the immune system.

Another problem is if a patient has a low endogenous expression of a certain receptor, i.e. the target molecule for the immunotherapy. There are also patients that become resistant during a therapy or where the receptors do not respond as in the beginning of the therapy.

Molecular engineering has improved the prospects for such antibody-based therapeutics, resulting in different constructs and humanized or human antibodies that can be administered more frequently.

The success of antibody treatment also varies depending on the availability of safe antibodies for the patient, the efficiency such as the number of binding sites on the target cell, and also the time of treatment. Apart from technical circumstances, the therapy is quite expensive. By using CpG ODNs, i.e. immunostimulatory oligonucleotides, to improve the responsiveness (e.g. induce/decrease cell surface molecules on the target cell) to immunotherapy of a patient, the immunotherapy could become more efficient and therefore also less expensive.

WO 01/97843 disclose a method for treating cancer by administrating to the subject an immunostimulatory nucleic acid and an antibody specific for the cell surface marker induced on the B cell in order to treat the cancer.

Regardless of the considerable effort spent on developing oligonucleotide based therapeutic approaches to cancer and inflammatory diseases etc, and the occasional success reported so far, there still remains a need for identifying new, more specific, functional and safe compounds and modes of administration, exhibiting improved efficacy and minimal or no side effects.

### SUMMARY

The present invention makes available a method for the regulation of certain cell surface antigens for increasing the efficiency of therapies related to cell surface antigens, such as immunotherapy for the prevention, treatment and/or alleviation of B-cell associated indications, such as cancer, inflammatory diseases and autoimmune diseases, such as MS.

The inventors surprisingly found that specific oligonucleotide sequences were capable of up-regulating the expression of certain cell surface molecules or cell surface antigens, in particular cell surface antigens, such as CD20, CD23, CD69 and CD80.

The use of immune modulators such as ODNs to increase the expression of certain cell surface antigens, could increase the efficiency of treatments that target the cell surface antigens, either alone or in different combinations. In particular, the inventors showed that the prior incubation with an oligonucleotide sequence according to the invention significantly increased the rate of apoptosis mediated by a monoclonal antibody (rituximab) administered to human B-cells. The inventors therefore make available a method for increasing the efficiency of treatments of various diseases (cancers, inflammatory diseases and autoimmune disorders) using a monoclonal antibody wherein the patient is preconditioned before the administration of said antibody, wherein the preconditioning comprises the administration of an oligonucleotide according to the invention. Other problems underlying the invention, as well as advantages associated with the invention, will become evident to the skilled person upon study of the description, examples, and claims, incorporated herein by reference.

### SHORT DESCRIPTION OF THE DRAWINGS

The invention will be described in closer detail in the following description, non-limiting examples and claims, with reference to the attached drawings in which

FIG. 1 consists of seven graphs (1a-1g) wherein FIG. 1a-d shows that treatment of CLL-cells with IDX-compounds increased the number of CD19+ cells expressing CD20, CD23, CD69 and CD80, as well as activation of NK cells. These effects are important steps in enhancing rituximab-induced ADCC of malignant B-CLL cells. FIG. 1e-g shows the cytokine profiles of IDX-compounds in PBMCs derived from CLL-blood.

FIG. 1a shows the effect of the test compounds on the expression of CD20 in peripheral blood mononuclear cells (PBMCs) from CLL patients. CLL-PBMCs were treated with medium only (untreated) or 1, 10 or 25 μM of IDX-compounds for 48 hrs and the surface expression of CD20 was subsequently analyzed by flow cytometry. Columns represent means of the mean fluorescence intensity (MFI) and standard deviations of CD19+/CD20+ cells from 18 patient samples.

FIG. 1b is a graph showing the effect of IDX-compounds on the expression of CD23 in malignant B-CLL cells. PBMCs isolated from fresh B-CLL blood were treated with medium only (untreated) or 1, 10 or 25 μM of IDX-compounds for 48 hrs and the surface expression of CD23 was subsequently analyzed by flow cytometry. Columns represent means of the MFI and standard deviations of CD19+/CD23+ cells from 18 patient samples.

FIG. 1c shows the effect of IDX-compounds on the expression of CD80 in malignant B-CLL cells. PBMCs isolated from fresh B-CLL blood were treated with medium only (untreated) or 1, 10 or 25 μM of IDX-compounds for 48 hrs and the surface expression of CD80 was subsequently analyzed by flow cytometry. Columns represent means of the MFI and standard deviations of CD80 on CD19+ cells from 18 B-CLL patient samples.

FIG. 1d shows the effect of IDX-compounds (1, 10 or 25 μM, or medium only, for 48 hrs) on the activation of NK-cells (CD3−/CD56+) in PBMCs from B-CLL cells and the surface expression of CD69 was subsequently analyzed by flow cytometry. Columns represent means of the percentages and standard deviations of CD3−/CD56+/CD69+ cells from 18 patient samples.

FIG. 1e shows human CLL-PBMCs treated with IDX-compounds for 48 hrs after which the expression of IL-6 was analyzed using the cytometric bead array (CBA) Flex kit (BD Biosciences, New Jersey, USA). Columns represent means of the expression in pg/ml and standard deviations in cells from 6 patient samples.

FIG. 1f shows the cytokine profiles of IDX-compounds in PBMCs derived from CLL-blood. Human CLL-PBMCs were treated with IDX-compounds for 48 hrs and the expression of IL-10 was measured by CBA analysis. Columns represent means of the expression in pg/ml and standard deviations in cells from 5 patient samples.

FIG. 1g shows the cytokine profiles of IDX-compounds in PBMCs derived from CLL-blood. Human CLL-PBMCs were treated with IDX-compounds for 48 hrs and the expression of IP-10 was measured by CBA analysis. Columns represent means of the expression in pg/ml and standard deviations in PBMC isolations from 4 patients.

FIG. 2 is a graph showing the increased CD20 expression on B cells in PBMCs isolated from MS patients (RRMS). The PBMCs were incubated for 48 hrs with 1, 10 or 25 μM of IDX9022. Cells were the harvested and analyzed for CD20 expression using FACS analysis. Columns represent means of MFI and standard deviations of CD19+/CD20+ cells from 4 patient samples.

FIG. 3 consists of six graphs showing that pretreatment of CLL-PBMCs with IDX-compounds enhances rituximab-mediated ADCC. In all diagrams, columns represent means of the percentages and standard deviations of apoptotic CD19+ cells.

FIG. 3a shows a graph where purified PBMCs from fresh CLL blood (n=20) were stimulated in vitro for 48 hrs with IDX9022 to increase CD19+/CD20+ levels, after which rituximab was added at 5 or 10 μg/ml. ADCC was measured by FACS analysis 24 hrs later, measuring the levels of Annexin V/7-AAD double positive CD19+ cells. The results are compared to those of untreated cells and cells treated with 1 and 10 μM of IDX9022 only. n=18.

FIG. 3b shows a graph where pre-treatment of CLL-PBMCs with rituximab before treatment with IDX9022 does not enhance rituximab-induced ADCC. Purified CLL-PBMCs were treated with rituximab at 5 or 10 μg/ml for 48 hrs and subsequently treated with 1 or 10 μM of IDX9022 for another 24 hrs. ADCC was analyzed as in FIG. 3a. n=10.

FIG. 3c shows a graph where PBMCs purified from fresh CLL blood were stimulated in vitro for 48 hrs with IDX9022 or IDX0150 to increase CD19+/CD20+ levels, after which rituximab was added at 5 or 10 μg/ml. ADCC was measured by FACS analysis 24 hrs later, measuring the levels of Annexin V/7-AAD double positive CD19+ cells. The results are compared to those of untreated cells and cells treated with IDXs only. n=10.

FIG. 3d shows a graph where PBMCs purified from fresh CLL blood were stimulated in vitro for 48 hrs with IDX9022 or IDX0505, respectively, to increase CD19+/CD20+ levels, after which rituximab was added at 5 or 10 μg/ml. ADCC was measured by FACS analysis 24 hrs later, measuring the levels of Annexin V/7-AAD double positive CD19+ cells. The results are compared to those of untreated cells and cells treated with 1 and 10 μM of the IDXs only. n=10.

FIG. 3e shows a graph where PBMCs purified from fresh CLL blood were stimulated in vitro for 48 hrs with IDX9022 or IDX9058 to increase CD19+/CD20+ levels, after which rituximab was added at 5 or 10 μg/ml. ADCC was measured by FACS analysis 24 hrs later, measuring the levels of Annexin V/7-AAD double positive CD19+ cells. The results are compared to those of untreated cells and cells treated with IDX-compounds only. n=10.

FIG. 3f shows a graph where pre-treatment of CLL-PBMCs with IDX0011 does not enhance rituximab-induced ADCC. PBMCs purified from fresh CLL blood were stimulated in vitro for 48 hrs with IDX9022 or IDX0011 to increase CD19+/CD20+ levels, after which rituximab was added at 5 or 10 μg/ml. ADCC was measured by FACS analysis 24 hrs later, measuring the levels of Annexin V/7-AAD double positive CD19+ cells. The results are compared to those of untreated cells and cells treated with IDX-compounds only. n=4.

FIG. 4 consists of three graphs (a-c) showing the expression of CD20, CD23 and CD80, respectively, on CD19+ cells after pulsed treatment with IDX-compounds IDX9022 (SEQ ID NO 1, Table 1) and IDX0150 (SEQ ID NO 7, Table 1).

FIG. 4a shows the results of CD20 expression on human CLL B-cells after varying time periods of treatment with IDX-compounds. PBMCs from four patients diagnosed with CLL were stimulated with 0.1, 1, 10 or 25 μM of IDX0150 and IDX9022 for 2, 6 or 24 hrs. Thereafter, cells were washed (w) to remove free IDX-compounds and the cells were incubated further for a total of 72 hrs. Some cells were incubated with IDX-compounds during the whole incubation period, i.e. 72 hrs, or with medium alone (untreated). Cells were subsequently harvested and analyzed for CD20 expression by flow cytometry. The mean percentages and standard deviations of CD20 positive cells out of CD19 positive cells are shown.

FIG. 4b is a graph showing the results of CD23 expression on CLL B-cells after varying time periods of treatment with IDX-compounds. PBMCs from one CLL patient were treated with IDX-compounds as in FIG. 4a. Cells were subsequently harvested and analyzed for CD23 surface expression by flow cytometry. The MFI of CD23 positive B-cells is shown.

FIG. 4c shows the results of CD80 expression in CLL B-cells after varying time periods of treatment with IDX-compounds. PBMCs from one CLL patient were stimulated with IDX-compounds as in FIG. 4a. Cells were subsequently harvested and analyzed for CD80 surface expression by flow cytometry. The MFI of CD80 positive B-cells is shown.

### DESCRIPTION

The following description is of the best mode presently contemplated for carrying out the invention. This description is not to be taken in a limiting sense, but is made solely for the purpose of describing the general principles of the invention. The scope of the invention should be determined with reference to the claims.

Before the invention is described in detail, it is to be understood that this invention is not limited to the particular compounds described or process steps of the methods described as such compounds and methods may vary. It is also to be understood that the terminology used herein is for purposes of describing particular embodiments only, and is not intended to be limiting. It must be noted that, as used in the specification and the appended claims, the singular forms “a,” “an” and “the” include plural referents unless the context clearly dictates otherwise. Thus, for example, reference to “a sequence” includes more than one such sequence, and the like.

Further, the term “about” is used to indicate a deviation of +/−2% of the given value, preferably +/−5% and most preferably +/−10% of the numeric values, when applicable.

For purposes of the invention, the term “immunomodulatory oligonucleotide” refers to an oligonucleotide as described above that induces an immune response either stimulating the immune system or repressing the immune system or both in an organism when administered to a vertebrate, such as a mammal.

The term “immunomodulatory response” describes the change of an immune response when challenged with an immunomodulatory oligonucleotide. This change is measurable often through the release of certain cytokines such as interleukins as well as other physiological parameters such as proliferation. The response can equally be one that serves to stimulate the immune system, as well as to repress the immune system depending on the cytokines induced by the immunomodulatory oligonucleotide in question.

The phrase “therapeutically effective amount” is used herein to mean an amount sufficient to induce the expression of said cell surface antigen in an amount that enhance the immunotherapy or enhance a response to other medicaments such as, steroids or other anti-inflammatory agents to some beneficial degree.

The term “diseases” includes but is not limited to: cancers, e.g. B-cell malignancies, lymphomas, leukemias, and conditions or diseases wherein suppression of B-cell immune function is therapeutically beneficial, e.g. autoimmune diseases (e.g. MS, thrombocytopenia, lupus or rheumatoid arthritis), allergic diseases and transplant rejections.

The invention finds utility in the treatment of cancer and MS, as supported by the in vitro data presented in the experimental section and illustrated in the attached figures.

An inflammatory disease is in this context defined as a disease characterized by inflammation. Examples include, but are not limited to, allergic conditions, asthma, allergic rhinitis, inflammatory bowel disease (Crohn's disease and related conditions), multiple sclerosis, chronic obstructive pulmonary disease (COPD), rheumatoid arthritis and cardiovascular diseases with an inflammatory component.

The term “inflammation” can be defined as an immunologic response to injury or irritation, characterized by local mobilization of white blood cells and antibodies, swelling and fluid accumulation. This is a response that is identical whether the injurious agent is a pathogenic organism, foreign body, ischemia, physical trauma, ionizing radiation, electrical energy or extremes of temperature. Although a defence and repair mechanism of the body, the reactions produced during inflammation may be harmful and develop into e.g. chronic inflammation, hypersensitivity reactions, systemic or local inflammatory diseases.

In order to make antibody drugs more efficient, an up-regulation of the specific antigen targets on the surface of the target cells. i.e., tumour cells might be helpful. One way of obtaining such an effect could be to stimulate the cells with immunomodulatory oligonucleotides. Immune stimulatory effects can be obtained through the use of synthetic DNA-based oligodeoxynucleotides (ODN). Such ODN have highly immunostimulatory effects on human and murine leukocytes, inducing B-cell proliferation; cytokine and immunoglobulin secretion; natural killer (NK) cell lytic activity and IFN-gamma secretion; and activation of dendritic cells (DCs) and other antigen presenting cells to express co-stimulatory molecules and secrete cytokines, especially the Th1-like cytokines that are important in promoting the development of Th1-like T-cell responses (Krieg et al, 1995). The increase in receptor density by ODNs could be mediated through a direct effect of the oligonucleotides on the cells, or through the induction of cytokines. An increase in antigen density or an increase in the population of cells expressing the target receptors would enable the antibodies to kill the tumour cells more efficiently, either through enhancing antibody-dependent cellular cytotoxicity (ADCC) or complement-dependent cytotoxicity (CDC).

The invention makes available novel oligonucleotide sequences according to any one of SEQ ID NO. 1-6 (see Table 1) capable of at least one of the following: induction of the expression of endogenous cytokines, such as but not limited to the interleukins IL-6 and IL-10, and/or up-regulation of the expression of specific cell surface antigens.

The invention also makes available the manufacture of a pharmaceutical composition comprising one of the oligonucleotides according to SEQ ID NO. 1-8, for the treatment or enhancement of the treatment comprising of up-regulation of the expression of a cell surface antigen, in order to treat or enhance a treatment of a condition wherein the antigen expressing cells participate in the pathogenesis of said condition.

TABLE 1
Examples of oligonucleotide sequences

| SEQ | | |
|---|---|---|
| ID | | |
| NO. | IDX-No | Seq 5′-3′ |
| 1 | IDX9022 | T*C*G*TCGTTCTGCCATCGTC*G*T*T |
| 2 | IDX9038 | T*C*G*TCGTTCGGCCGATCG*T*C*C |
| 3 | IDX9052 | G*G*G*GTCGTCTG*C*G*G |
| 4 | IDX9058 | G*A*T*CGTCCGTCGG*G*G*G |
| 5 | IDX9071 | T*C*G*TTCGTCTGCTTGTTC*G*T*C |
| 6 | IDX0011 | C*C*G*GGGTCGCAGCTGAGCCCA*C*G*G |
| 7 | IDX0150 | G*G*A*ACAGTTCGTCCAT*G*G*C |
| 8 | IDX0505 | G*G*A*A*C*A*G*T*T*G*C*T*C*C*A*T*G*G*C |

*= phosphorothioate modification

The above sequences SEQ ID NO. 1-6 have been designed by the inventors, and are to the best knowledge of the inventors, not previously known.

SEQ ID NO. 7 (IDX0150) is known from U.S. Pat. No. 6,498,147, and currently undergoing clinical trials for the treatment of inflammatory bowel disease (Kappaproct®, Index Pharmaceutical AB, Solna, Sweden).

SEQ ID NO. 8 (IDX0505) is known from WO 2007/004977 and WO 2007/004979 (as IDX0526) and corresponds to SEQ ID NO. 7 (IDX0150) but comprises a GC motif instead of a CG motif.

SEQ ID NO. 7, although known for medical use, is to the best knowledge of the inventors not previously known for use in the induction of cell surface antigens.

SEQ ID NO. 8, although known for medical use, is to the best knowledge of the inventors not previously known for use in the induction of cell surface antigens.

The inventors have surprisingly shown that specific oligonucleotides are capable of eliciting or increasing the expression of specific cell surface markers, here exemplified by CD20, CD23, CD69 and CD80.

Consequently, the present inventors make available an isolated and substantially purified oligonucleotide chosen among SEQ ID NO. 1-6.

According to one embodiment, at least one nucleotide in such oligonucleotides has a phosphate backbone modification. Preferably said phosphate backbone modification is a phosphorothioate or phosphorodithioate modification.

The inventors also make available pharmaceutical compositions comprising an oligonucleotide according to any one of SEQ ID NO. 1-6. Said pharmaceutical compositions further preferably comprise a pharmacologically compatible and physiologically acceptable excipient or carrier, chosen from saline, liposomes, surfactants, mucoadhesive compounds, enzyme inhibitors, bile salts, absorption enhancers, cyclodextrins, or a combination thereof.

The invention finds utility in treatments or in the enhancement of treatments wherein the oligonucleotides are used as a tool for up-regulating a specific cell surface antigen on a specific cell type and in which specific antibodies are administered to bind to the up-regulated cell surface antigens for the purpose of eliminating a specific cell type.

Another embodiment of the invention is thus the use of an isolated and substantially purified oligonucleotide according to any one of SEQ ID NO. 1-6, and 7-8 for the manufacture of a pharmaceutical composition for the treatment, and/or enhancement of antibody based therapies.

Yet another embodiment of the invention is the use of an isolated and substantially purified oligonucleotide according to any one of SEQ ID NO. 1-8, and 7-8 for the manufacture of a pharmaceutical composition for up-regulation of the expression of a cell surface antigen, chosen between CD20, CD23, CD69 and CD80, in order to treat or enhance a treatment of a condition wherein the antigen expressing cells participate in the pathogenesis of said condition. Preferably the antigen is CD20.

Another embodiment is a pharmaceutical composition comprising one of the oligonucleotides according to SEQ ID NO. 1 [IDX9022] or SEQ ID NO. 4 [IDX9058], preferably SEQ ID NO. 1 [IDX9022].

Another embodiment is the use of an isolated and substantially purified oligonucleotide according to SEQ ID NO. 1, SEQ ID NO. 4, SEQ ID NO. 7 for the manufacture of a pharmaceutical composition for the up-regulation of cell surface antigens, wherein the antigens are chosen from but not limited to CD20, CD69 or CD80.

Another embodiment is the use of an isolated and substantially purified oligonucleotide according to SEQ ID NO. 1, SEQ ID NO. 2, SEQ ID NO.5 or SEQ ID NO.7 for the manufacture of a pharmaceutical composition for the up-regulation of cell surface antigens, wherein the antigens are chosen from but not limited to CD23.

One embodiment is the use of an isolated and substantially purified oligonucleotide according to SEQ ID NO. 7 [IDX0150] or SEQ ID NO. 8 [IDX0505], for the manufacture of a pharmaceutical composition for up-regulation of the expression of the cell surface antigen CD20 in order to treat or enhance a treatment of a condition wherein CD20 expressing cells participate in the pathogenesis of said condition.

Yet another embodiment of the invention is the use of an isolated and substantially purified oligonucleotide for the manufacture of the above mentioned pharmaceutical composition, wherein the oligonucleotide according to SEQ ID NO. 1-8, is administered in an amount effective to induce the endogenous production of at least one cytokine, and the up-regulation of the expression of one of the chosen cell surface antigens, preferably the antigen is CD20.

The inventors therefore make available, as one embodiment of the invention, compounds and methods for the treatment of said diseases wherein the inventive compounds presented in Table 1 are used either alone; to increase the expression of endogenous cytokines, such as the interleukins IL-6 and IL-10, but not limited to these, or to up-regulate the expression of one or more of the cell surface antigens CD20, CD23, CD69 and CD80; or in combination with an anti-cancer therapy, anti-MS therapy or other treatments, preferably an immunological treatment comprising the administration of an antibody to the patient.

Another embodiment is the use of an isolated and substantially purified oligonucleotide according to SEQ ID NO. 1, for the manufacture of a pharmaceutical composition for the up-regulation of cell surface antigens, wherein the antigens are chosen from but not limited to CD20, CD80 and/or CD69.

Without wishing to be bound to any specific theory, the inventors contemplate that the effect of the inventive compounds at least in part is accountable to their capability to induce endogenous production of cytokines. Consequently, one embodiment of the invention involves the administration of an oligonucleotide according to SEQ ID NO.1-8 in an amount effective to induce endogenous production of cytokines, such as but not limited to IL-6 and/or IL-10.

The invention also make available a method for the treatment, and/or enhancement of a condition wherein CD20 expressing cells participate in the pathogenesis of said condition, and/or enhancement of said treatment, wherein one of the described oligonucleotides according to SEQ ID NO. 1-8 is administered in a dose sufficient to induce the up-regulation of the cell surface antigen CD20.

The inventive compounds SEQ ID NO. 1-6, and 7-8, presented in Table 1, offer a possibility to increase the efficiency of antibody based treatments to different diseases such as cancers, inflammatory and autoimmune disorders, either alone, or preferably in the form of a preconditioning before the administration of another treatment, e.g. the administration of a monoclonal antibody.

The diseases are chosen from but not limited to: cancer; (e.g. B-cell malignancies, lymphomas or leukemias) and conditions or diseases wherein suppression of B-cell immune function is therapeutically beneficial, e.g. autoimmune diseases (e.g. MS, thrombocytopenia, lupus or rheumatoid arthritis) allergic diseases, transplant rejection (and indications where other therapeutic regimens involving administration of antigenic moieties, e.g., gene, protein or cell therapy, are used.)

In one embodiment, the condition to be treated is cancer, and one of the above described oligonucleotides, chosen between SEQ ID NO. 1-6, and 7-8 is administered in a dose effective to induce the endogenous production of at least one cytokine and the up-regulation of the expression of the cell surface antigen CD20.

In yet another embodiment, the condition to be treated is multiple sclerosis, and one of the above described oligonucleotides, chosen between SEQ ID NO. 1-6, and 7-8 is administered in a dose effective to induce the endogenous production of at least one cytokine and the up-regulation of the expression of the cell surface antigen CD20.

The oligonucleotides according to the invention can be delivered subcutaneously or topically on a mucous membrane. The term “topically on a mucous membrane” includes oral, gastric, pulmonary, rectal, vaginal, and nasal administration. It is well known that the accessibility and vascular structure of the nose make nasal drug delivery an attractive method for delivering both small molecule drugs and biologics, systemically as well as across the blood-brain barrier to the CNS. The nucleotides can be delivered in any suitable formulation, such as suitable aqueous buffers, e.g. but not limited to phosphate buffered saline (PBS). It is contemplated that the nucleotides are administered in a suitable formulation, designed to increase adhesion to the mucous membrane, such as suitable gel-forming polymers, e.g. chitosan etc; a formulation enhancing the cell uptake of the nucleotides, such as a lipophilic delivery vehicle, liposomes or micelles; or both.

Preferably the route of administration of said medicament is chosen from intravenous, subcutaneous, mucosal, intramuscular, and intraperitoneal administration. The mucosal administration is chosen from nasal, oral, gastric, ocular, rectal, urogenital and vaginal administration.

The oligonucleotide is administered in a single dose or in repeated doses. The currently most preferred embodiment entails one single dose of the nucleotide according to the invention, administered i.v., or s.c., or to a mucous membrane, e.g. given intranasally, orally, rectally or intravaginally.

According an embodiment, the oligonucleotide is administered by intravenous injection or infusion.

According to another embodiment the oligonucleotide is administered subcutaneously to a patient in need thereof.

The oligonucleotide is administered in a therapeutically effective dose. The definition of a “therapeutically effective dose” is dependent on the disease and treatment setting, a “therapeutically effective dose” being a dose which alone or in combination with other treatments results in a measurable improvement of the patient's condition.

The phrase “therapeutically effective amount” or “therapeutically effective dose” is used herein to mean an amount sufficient to enhance a response to a treatment.

According to one embodiment the oligonucleotide is administered in an amount of about 1 μg to about 2000 μg per kg body weight. Preferably the oligonucleotide is administered in an amount of about 10 μg to about 1000 μg per kg body weight. Most preferably, the oligonucleotide is administered in an amount of about 20 μg to about 600 μg per kg body weight.

According to another embodiment, the invention is used as a pre-treatment before an antibody-based treatment of a B-cell related disease wherein a pharmaceutical composition is administered to a patient.

One embodiment of the invention is to supply a pharmaceutical composition capable of increasing the therapeutic effect of a cell surface antigen targeted therapy, wherein said CD target(s) is chosen among but not limited to CD20, CD23, CD69 and CD80, preferably CD20.

One embodiment of the above described method entails increasing the therapeutic effect of a cell surface antigen targeted therapy, wherein said target is CD20, and wherein an oligonucleotide according to one SEQ ID NO. 1-8, is administered in a dose sufficient to induce the up-regulation of the cell surface antigen CD20.

One embodiment of the described method is to administer the oligonucleotide before, or essentially simultaneously with a treatment.

In one embodiment said treatment includes the administration of an antibody, preferably a CD20-antibody.

One embodiment of the invention is to pre-examine a patient's expression of certain cell surface antigens, as well as the response to said nucleotides and by that “predicting” the efficiency of a treatment and also be able to improve the treatment by giving the patient a composition as described above.

One embodiment of the invention is therefore a method of increasing the efficiency of an immunotherapy directed towards a specific antigen target, wherein the method comprises the steps of:
a) collection of a sample from said patient, i.e. tissue (blood, biopsy, etc) and quantification of the expression of the antigen of interest in said sample.
b) addition of one oligonucleotide chosen from SEQ ID NO. 1-8 in Table 1, to said sample.
c) determination whether or not the expression of the antigen can be up-regulated in the sample by the addition of said oligonucleotide;
d) depending on the outcome of step c), administration of said oligonucleotide to said patient in an amount effective to up-regulate the expression of CD20, CD23, CD69 and/or CD80, preferably CD20.
e) administration of an antibody drug to said patient, wherein said antibody drug is directed to the antigen of interest.

Preferably said antibody is an antibody directed to CD20

According to an embodiment, the above described method is used to increase the efficiency of an immunotherapy directed towards a specific antigen target, wherein the disease is cancer or multiple sclerosis.

The phrase “cell surface antigen”, “target” or “receptor” is an antigen expressed on the surface of a target cell or B-cell, which can be targeted with an antagonist which binds thereto.

The target cells are preferably B-cells but are not limited thereto.

Examples of cell surface antigens include but are not limited to; CD20, CD23, CD69 or CD80.

It is an embodiment of the invention to provide novel methods for increasing the level of CDs preferably before the administration of a CD-antibody.

Another embodiment is the up-regulation of cell surface antigens, e.g. CD20, CD23, CD69 and/or CD80, as a pre-conditioning or adjunct therapy. It is contemplated that the up-regulation of specific cell surface antigens would increase the efficacy of antibodies directed towards these antigens, such as rituximab (anti-CD20), lumiliximab (anti-CD23) and galiximab (anti-CD80).

Examples of presently available, or under evaluation, antibodies include, but are not limited to, rituximab (Rituxan®, MabThera®), ocrelizumab, veltuzumab, ofatumumab, tositumomab, ibritumomab, lumiliximab, alemtuzumab (Campath®, MabCampath®), galiximab, epratuzimab, bevacizumab (Avastin®), and trastuzumab (Herceptin®).

The treatment is preferably an immunological therapy involving the administration of an antibody to the patient. Examples of antibodies include antibodies currently in use as well as under evaluation, e.g. rituximab, ocrelizumab, altuzumab, ofatumumab, tositumomab, ibritumomab (all directed to CD20), lumiliximab (anti-CD23), alemtuzumab (anti-CD52), galiximab (anti-CD80), epratuzimab (anti-CD22), and daclizumab (anti-CD25).

In a further embodiment, the oligonucleotides of the invention can be coupled to a so called “delivery molecule” which imparts a specific cellular uptake or targeting property to the attached immunomodulatory oligonucleotides.

Commonly used examples of such include but are not limited to hydrophobic molecules like cholesterol functional groups, specific peptides that have an increased ability to translocate cellular membranes such as cationic antimicrobial peptides or commonly recognized protein transduction domains (PTDs) or DNA vectors.

When given in combination with an immunotherapy, the inventive compounds are preferably administered in advance of said immunotherapy, preferably about 6, about 12, about 24, or about 48 hours in advance of the therapy. The inventive compounds may also be administered longer before the therapy, for example about 3 days, or about 5, 7, or 14 days before said therapy.

When given in combination with an immunological therapy, and in particular a therapy involving the administration of an antibody, the inventive compound is preferably administered before the administration of the antibody to the patient, and most preferably sufficiently before in order to allow for the up-regulation of cell surface molecules or cell surface markers towards which the specific antibody is targeted. The treatment is preferably an immunological therapy involving the administration of an antibody to the patient.

Another embodiment of the invention would be to repeat the pre-treatment with oligonucleotides according to the invention to boost the effect and thereby increasing the efficacy of an immunotherapy further.

A preferred embodiment of the invention comprises the use as defined above, wherein antibody-therapy is administered before, after or essentially simultaneously with the administration of said oligonucleotide. This treatment is chosen among immunological therapy, treatment with antibodies, steroids, cortisone treatment, interferon treatment, or a combination of any of these.

Consequently the present invention also comprises a method for the treatment of said diseases and disorders wherein an isolated oligonucleotide sequence according to any one of the sequences presented in Table 1 (SEQ ID NO. 1-8) is administered to a patient in need thereof.

In any one of the above embodiments of the invention, said oligonucleotide is administered in a dose effective to elicit or increase or up-regulate the expression of at least one cell surface molecule or cell surface antigen, in particular a cell surface marker chosen among CD20, CD23, CD69 and CD80.

A pharmaceutical composition, wherein the oligonucleotide is chosen from one of SEQ ID NO. 1, SEQ ID NO. 4 and SEQ ID NO. 7 and SEQ ID NO. 8 [IDX9022; IDX9058; IDX0150; IDX0505].

The use of the pharmaceutical composition, wherein the oligonucleotide is SEQ ID NO. 1 [IDX9022].

Another embodiment of the invention is a method for the treatment, and/or enhancement of a treatment, wherein an oligonucleotide according to SEQ ID NO. 1-8 (Table 1) is administered in a dose effective to induce the endogenous production of at least one cytokine and the up-regulation of the expression of one or more of the cell surface antigens CD20, CD23, CD69 and CD80.

Another embodiment of the invention is a method for the treatment, and/or enhancement of a treatment, wherein a pharmaceutical composition containing any one of the oligonucleotides presented in Table 1, wherein the composition is administered to a patient.

CD targeted therapies are treatments that include antibody treatments, as well as up/down regulation of CD antigens at the cell surface. A skilled person is well aware of the fact that there are several approaches to the treatment of B-cell associated diseases. Naturally new approaches are constantly being developed, and it is conceived that the oligonucleotides, their use and methods of treatment according to the present invention, will find utility also in combination with future treatments. The inventive oligonucleotides will have utility in combination with existing or future immunotherapies.

The embodiments of the invention have many advantages. So far, the administration of an oligonucleotide in the doses defined by the inventors has not elicited any noticeable side-effects. Further, the mucosal administration is easy, fast, and painless, and surprisingly results in a systemic effect. It is held that this effect, either alone, or in combination with existing and future treatments of said diseases, offers a promising approach to fight these diseases as well as related diseases.

### EXAMPLES

### 1. The Effects of IDX-Compounds on the Surface Expression of CD20 on PBMCs from Chronic Lymphocytic Leukemia (CLL) Patients and Combination Treatment of CLL Cells with IDX-Compounds Followed by Rituximab

### Materials and Methods

### Test Compounds

In total, 8 IDX-compounds (SEQ ID NO. 1-8 Table 1) were investigated for their effects on the cell surface expression of CD20, CD23 and CD80 on CD19+ cells in PBMCs isolated from CLL patients. All IDX-compounds were also investigated for their effect on activation of NK-cells by studying the expression of CD69 on CD3−/CD56+ cells. All IDX-compounds were synthesized by Biomers.net (Ulm, Germany) except IDX0150 which was ordered from Avecia (Massachusetts, USA).

### Formulation

The IDX-compounds were adjusted with phosphate buffered saline (PBS, Invitrogen, Carlsbad, Calif.) to reach a stock concentration of 500 μM by aid of UV spectrophotometry (SmartSpec® 3000, BIO-RAD, Hercules, USA) and stored at −20° C. until used.

### Cells

Heparinized peripheral blood was obtained after informed consent from patients diagnosed with B-CLL with significant circulating disease. All patients were diagnosed by routine immunophenotypic, morphologic and clinical criteria.

The mononuclear cell fraction was isolated by Ficoll-Hypaque (Seromed, Berlin, Germany) gradient centrifugation. The isolated cells were immediately incubated at 37° C. in RPMI-medium supplemented with 10% FCS, 1% PenStrep, 2 mM L-glutamine, 10 mM HEPES and 1 mM Sodium Pyruvate.

### Antibodies and Chemicals

Fluorochrome conjugated (Allophycocyanin (APC), R-phycoerythrin (PE)) antibodies, mouse anti-human CD19-PE-Cy7, CD20-APC-Cy7, CD23-APC, CD80-PE, CD3-APC, CD56-PE and CD69-PE-Cy7 and isotypes IgG1,κ-PE-Cy7 and IgG2b,κ-APC were obtained commercially from BD Biosciences (New Jersey, USA).

### Cell Treatment

Freshly isolated CLL cells were treated with 1, 10 and 25 μM of each of 8 different IDX-compounds in 500 μl of assay medium at a concentration of 2×10⁶ cells/ml in 48-well plates, or in 200 μl in 96-well plates.

### FACS Analysis

After 48 hrs of incubation with the IDX-compounds, 200 μl of the cells were spun down in 96-well plates, re-suspended in 100 μl of 2% FCS (in PBS) and incubated with two sets of mixed fluorochrome conjugated antibodies against CD19, CD20, CD23, CD69, CD80, CD3 and CD56, for 30 min at 4° C. The cells were then washed twice in pure PBS and subsequently analyzed by FACS using a FACSArray bioanalyzer for surface antigen expression analysis.

Before incubation, a fraction of the freshly isolated CLL cells were stained with the two previously described antibody mixes for direct analysis of surface antigen expression by FACS.

### ADCC Assay

For combinatory treatment cells were treated with IDX0011, IDX9022, IDX9058, IDX0150 or IDX0505 for 48 hrs. The cells were then washed twice with PBS and rituximab (Roche Pharmaceuticals) was added at a final concentration of 5 or 10 μg/ml. The cells were incubated for 30 min at 37° C. whereafter a F(ab′)2 fragment (Jackson Immunoresearch, Baltimore, USA) was added as a crosslinker and the cells were incubated again at 37° C. After 24 hrs the cells were harvested for apoptosis analysis by FACS.

CDC Assay

For combinatory treatment cells were treated with the above mentioned IDX-compounds for 48 hrs. The cells were then washed twice with PBS and incubated for 4 hrs with 30% human serum and rituximab (at 5 or 10 μg/ml) in RPMI media. Heat-inactivated human serum was used as control wells. After 4 hrs cells were harvested for apoptosis analysis by FACS.

### Apoptosis Assay

After 3 days from day 0, the cells were harvested for apoptosis analysis. The cells were spun down in 96-well plates, re-supended in 2% FCS as above and incubated with an antibody mix of CD19 and CD3 (BD Biosciences) for 30 min at 4° C. The cells were washed twice with PBS and subsequently stained with Annexin V and 7-AAD (BD Biosciences) for 10 min at RT for analysis of early and late apoptosis, respectively. The cells were analyzed by flow cytometry as above.

### Cytometric Bead Array

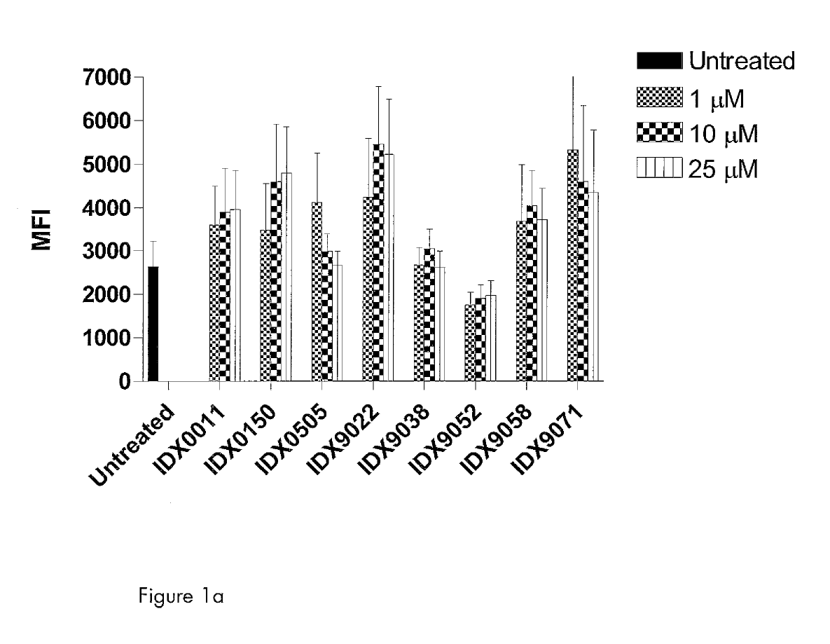

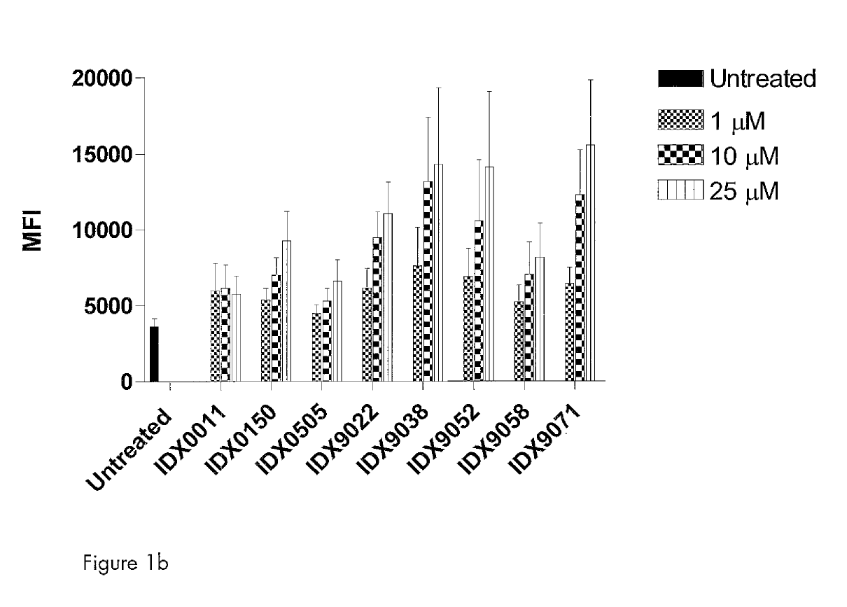

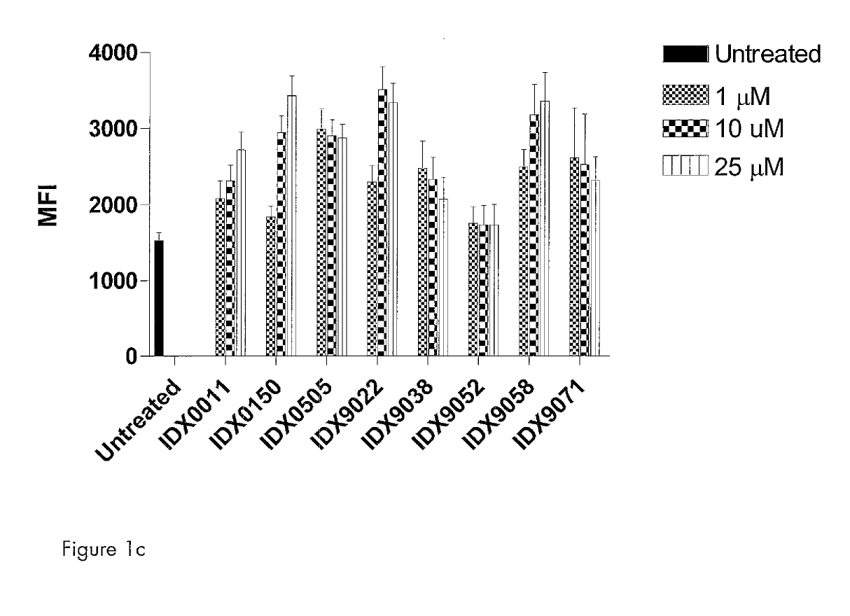

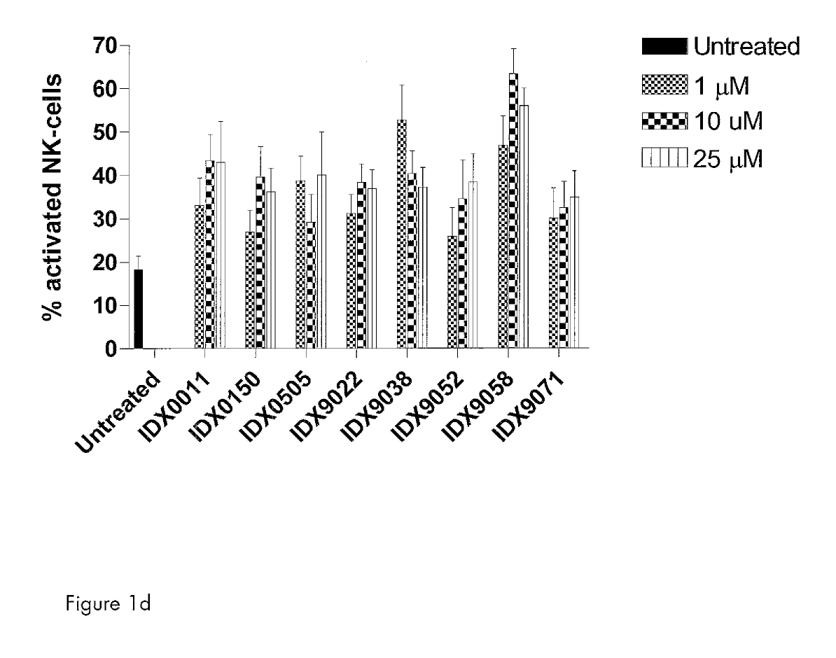

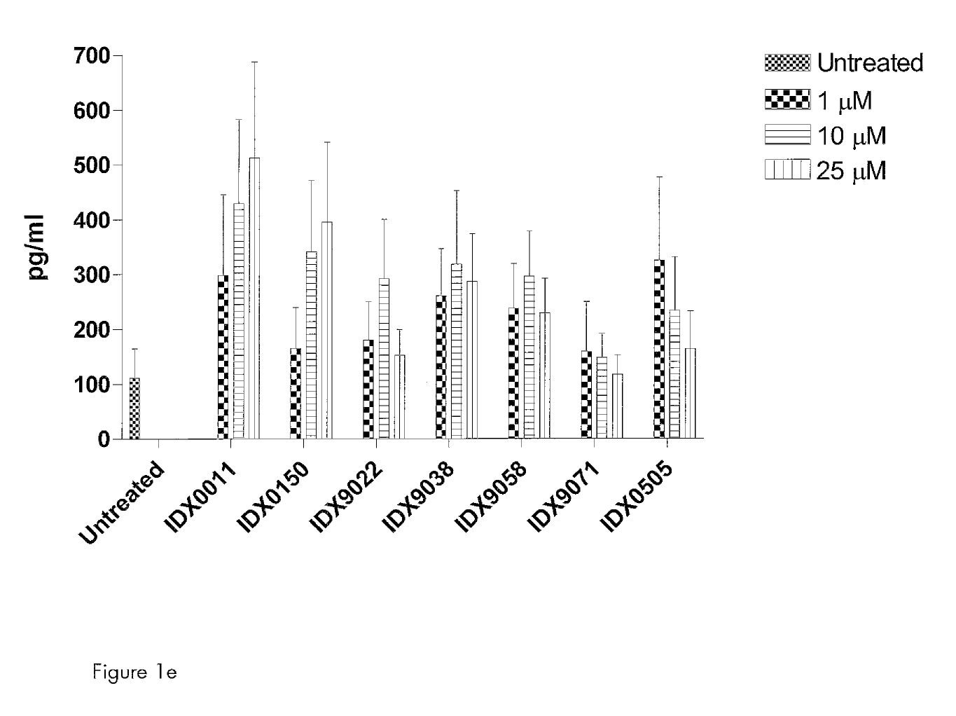

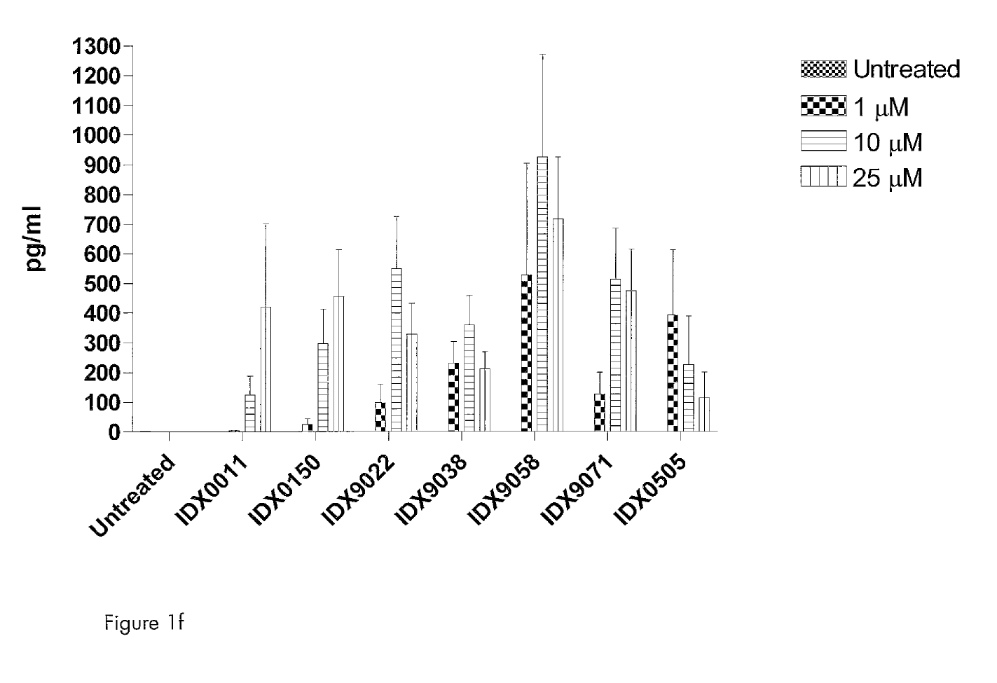

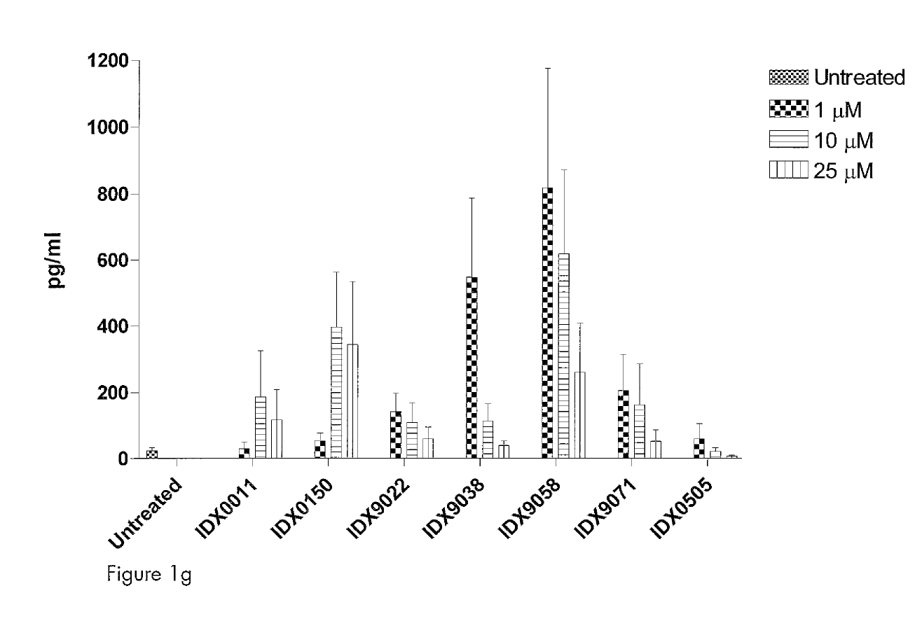

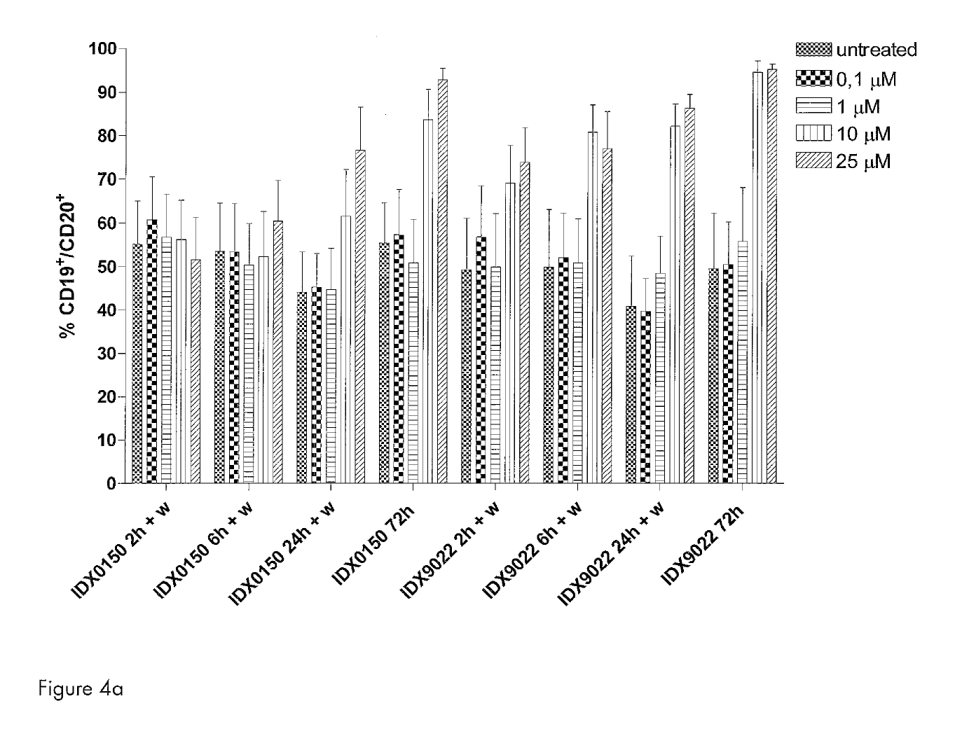

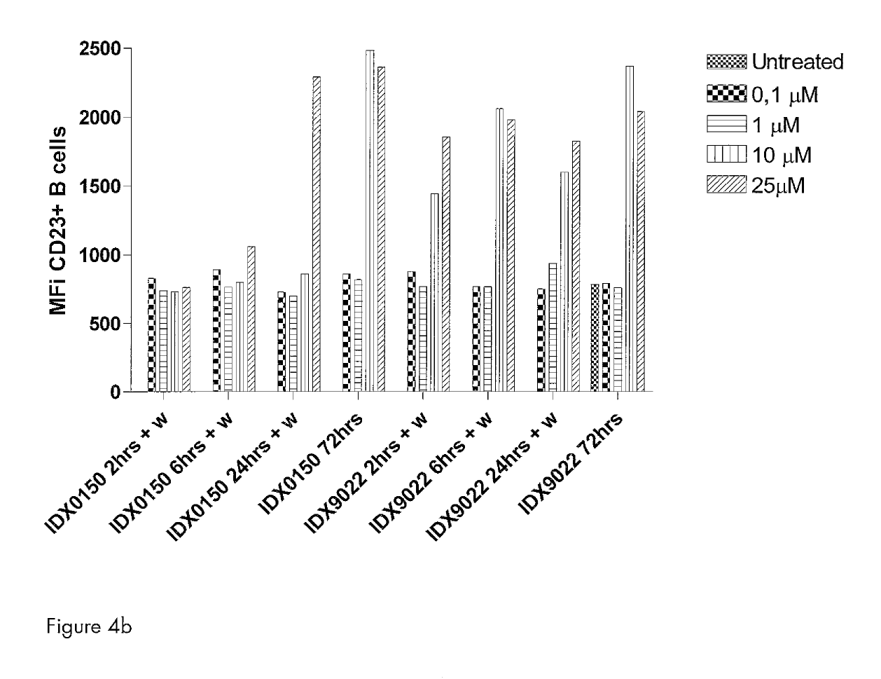

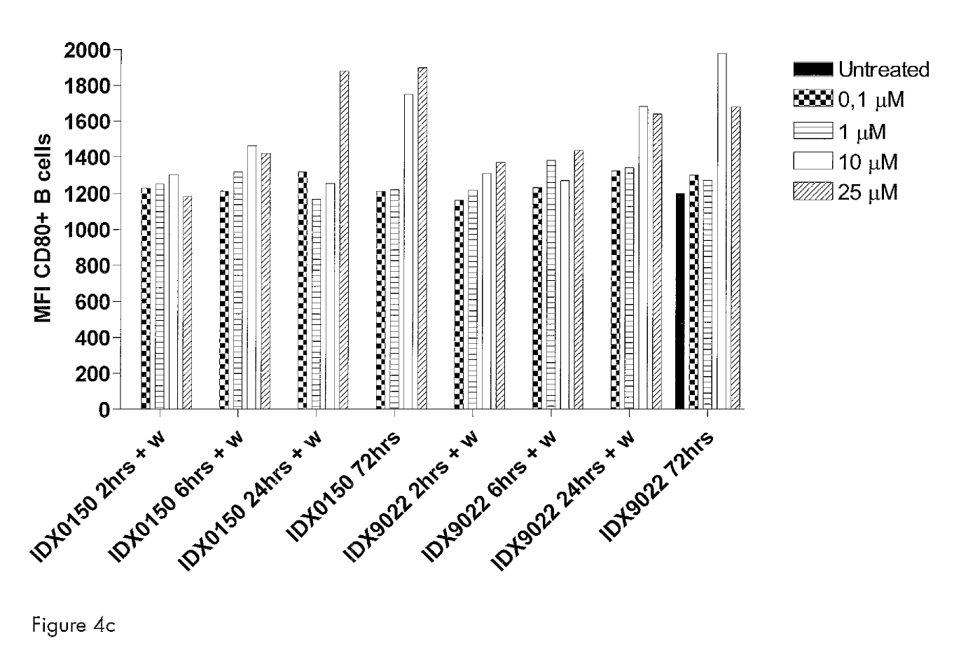

Human CLL-PBMCs were treated with IDX-compounds for 48 hrs and the expression of cytokines was analyzed using the cytometric bead array (CBA) Flex kit (BD Biosciences). Columns represent means of the expression in pg/ml and standard deviations in cells from 6 patients in FIG. 1e, 5 patients in FIG. 1f and 4 patients in FIG. 1g.

IL-6, IL-10, TNF-α, IFN-γ and IP-10 cytokines were measured utilizing the CBA Flex kit, according to the manufacturer's protocol. The samples were analyzed using a FACSArray flow cytometer and subsequently quantified using the FCAP Array software (BD Biosciences). The lower detection limit was 20 pg/ml for each cytokine.

### Results

CpG oligonucleotides affect innate and adaptive immune responses including antigen presentation, co-stimulatory molecule expression and induction of cytokines, which makes them interesting therapeutic tools for use in different disease contexts. Most of the information on the immunobiology of CpG oligonucleotides has been derived from studies with human PBMCs or mouse spleen cells, which constitute reliable sources of large numbers of immune cells to use as model systems. In this study we investigated the effect of IDX-compounds on the expression of CD20 on B-cells using PBMCs from patients diagnosed with CLL.

In order to make antibody drugs more efficient, an up-regulation of the specific antigen targets on the surface of tumor cells could be helpful. One way of obtaining such an effect could be to stimulate the cells with CpG oligonucleotides. CpG-ODNs have highly immunostimulatory effects on human and murine leukocytes, inducing B-cell proliferation; cytokine and immunoglobulin secretion; natural killer (NK) cell lytic activity and IFN-gamma secretion. In addition, CpG-ODNs activate dendritic cells (DCs) and other antigen presenting cells, leading to expression of co-stimulatory molecules and secretion of cytokines, especially the Th1-like cytokines that are important in promoting the development of Th1-like T cell responses (Krieg et al, 1995 and 2006). The increase in receptor density by CpG-ODNs could be mediated through a direct effect of the oligonucloetides on the cells, or through the induction of cytokines. An increase in antigen density or an increase in the population of cells expressing the target receptors would enable the antibodies to kill the tumor cells more efficiently, either through enhancing antibody-dependent cellular cytotoxicity (ADCC) or complement-dependent cytotoxicity (CDC).

Treatment of the cells with IDX-compounds increased the number of CD19+ cells expressing CD20 as well as the mean fluorescence intensity (MFI) of CD20, i.e. the amount of CD20 expressed per cell (FIG. 1a). The largest increase of CD20 expression was induced by IDX-compounds IDX9022 (SEQ ID NO. 1, Table 1), IDX9071 (SEQ ID NO. 5, Table 1) and IDX0150 (SEQ ID NO. 7, Table 1) (FIG. 1a). IDX0505 (SEQ ID NO. 8, Table 1), which instead of a CG contains a GC, also induced CD20 expression but at lower levels (FIG. 1a).

For most of the IDX-compounds tested there was a dose-response induction of CD20, with the most enhancement observed at 10 or 25 μM, and less induction at 1 μM (FIG. 1a). The IDX-compounds that were shown to be most efficient in up-regulating CD20 all have in common that they are potent inducers of IL-6 and IL-10 (FIGS. 1e and f). IL-6 and IL-10 are released from activated B-cells in response to IDX-compounds, and this indicates that IDX-compounds that are potent activators of B-cell cytokine release also are efficient in up-regulating CD20.

Treatment of the cells with IDX-compounds increased the number of CD19+ cells expressing CD23 as well as the MFI of CD23 (FIG. 1b). The largest increase of CD23 expression was induced by IDX-compounds IDX9038 (SEQ ID NO. 2, Table 1), IDX9052 (SEQ ID NO. 3, Table 1) and IDX9071 (SEQ ID NO. 5) (FIG. 1b). The oligonucleotide IDX0505 (SEQ ID NO. 8, Table 1), which instead of a CG contains a GC, was a poor inducer of CD23 (FIG. 1b).

For most of the IDX-compounds tested there was a dose-response induction of CD23, with the highest up-regulation observed at 25 μM, and less induction at 1 and 10 μM (FIG. 1b). As for CD20, the IDX-compounds that were shown to be most efficient in up-regulating CD23 all have in common that they are potent inducers of IL-6 and IL-10 (FIGS. 1e and f). In addition, IDX9038 and IDX9052 also induce IFN-alpha which is known to inhibit B-cells proliferation in vitro. The production of IL-6 and IL-10 might abolish this effect.

Treatment of CLL cells with IDX-compounds enhance the expression of CD23 on the cell surface of CD19+B-cells, which subsequently could enhance apoptosis of B-CLL cells induced by lumiliximab.

CD80 (also referred to as B7-1) is an important immune accessory molecule expressed on antigen presenting cells. Phenotypic studies of human CLL cells demonstrated that CLL cells express little or no CD80 (data not shown).

Treatment of the CLL cells with IDX-compounds increased the number of CD19+ cells expressing CD80 as well as the MFI of CD80 (FIG. 1c). The largest increase of CD80 expression was induced by IDX-compound IDX9058 (SEQ ID NO. 4, Table 1) (FIG. 1c). The IDX-compounds IDX0150 and IDX0505 (SEQ ID NO. 7 and 8, Table 1) also induced considerable amounts of CD80 expression (FIG. 1c).

For most of the IDX-compounds tested there was a dose-response induction of CD80 in B-cells from CLL patients, with the most enhancement observed at 10 or 25 μM, and less induction seen at 1 μM (FIG. 1c). In addition, the IDX-compounds that were shown to be most efficient in up-regulating CD80 on the CLL B-cells all have in common that they are potent inducers of IL-6 and IL-10 (FIGS. 1e and f) in accordance with the results presented in example 1.

Galiximab seems to induce apoptosis of tumor cells through mechanisms similar to rituximab. Thus, there is good reason to believe that an up-regulation of CD80 surface expression might enhance galiximab-induced apoptosis of B-CLL cells.

All IDX-compounds induced activation of NK-cells as seen by an increased number of CD3−/CD56+ cells expressing CD69 (FIG. 1d). The strongest activation of NK cells was observed byIDX-compounds IDX9022 (SEQ ID NO. 1, Table 1) IDX9038 (SEQ ID NO. 2), IDX9058 (SEQ ID NO. 4, Table 1) as well as IDX0150 (SEQ ID NO. 7, Table 1) and IDX0505 (SEQ ID NO. 8, Table 1).

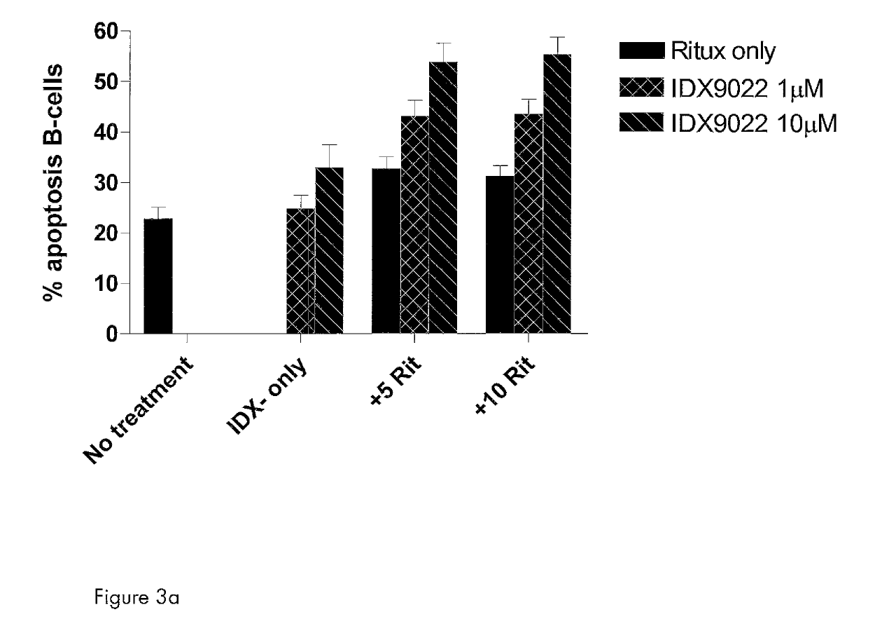

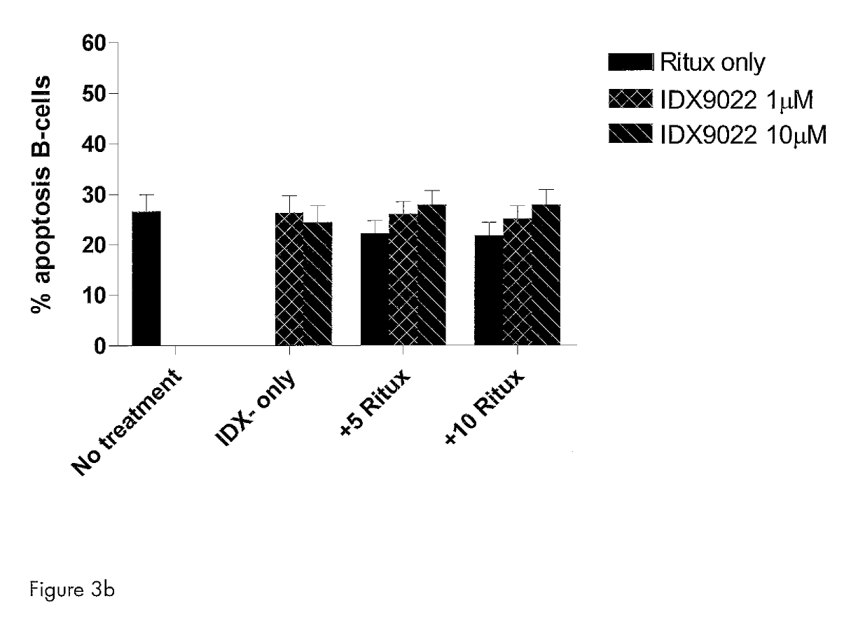

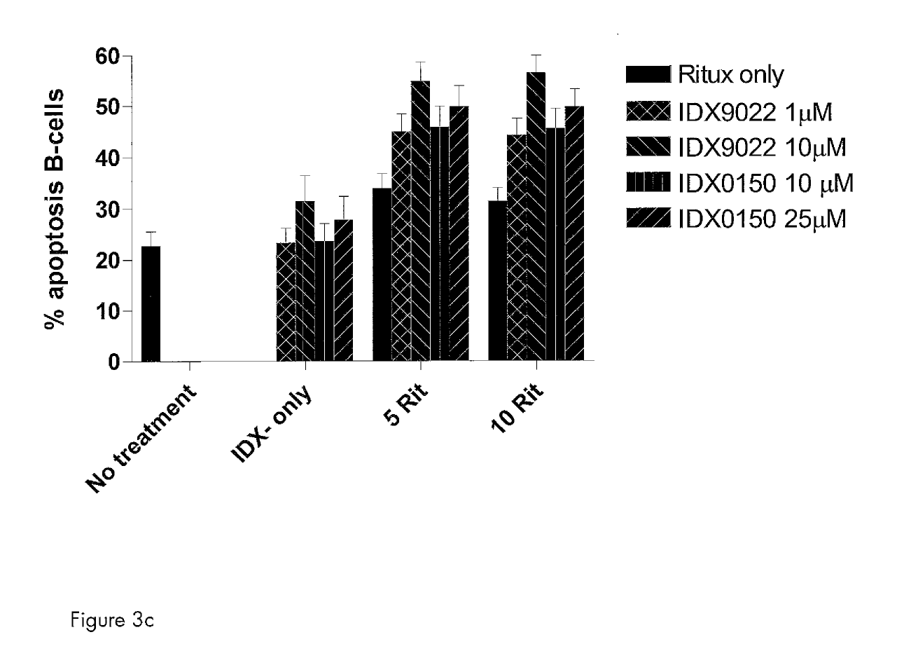

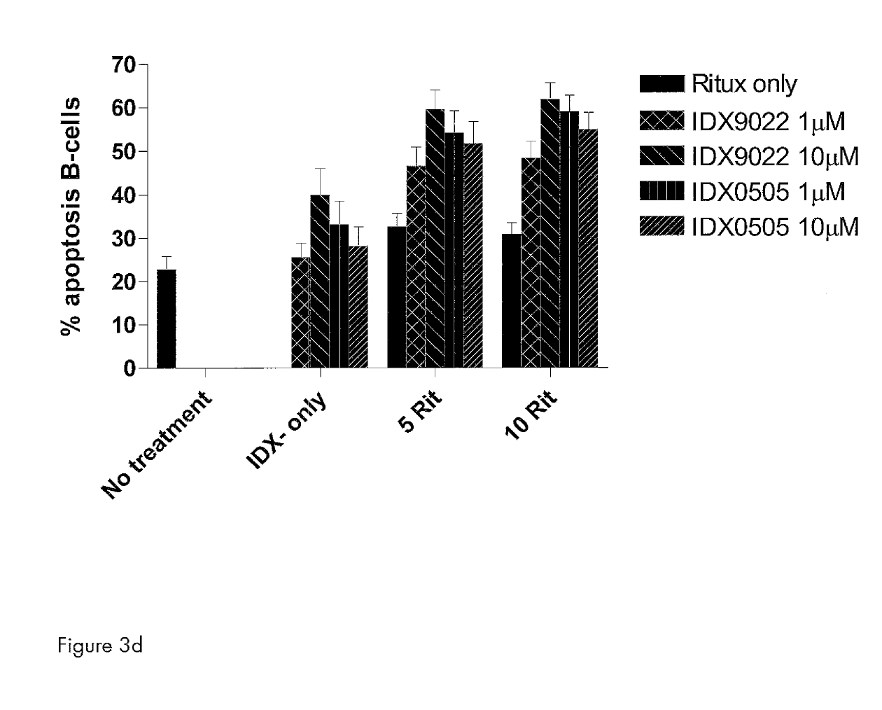

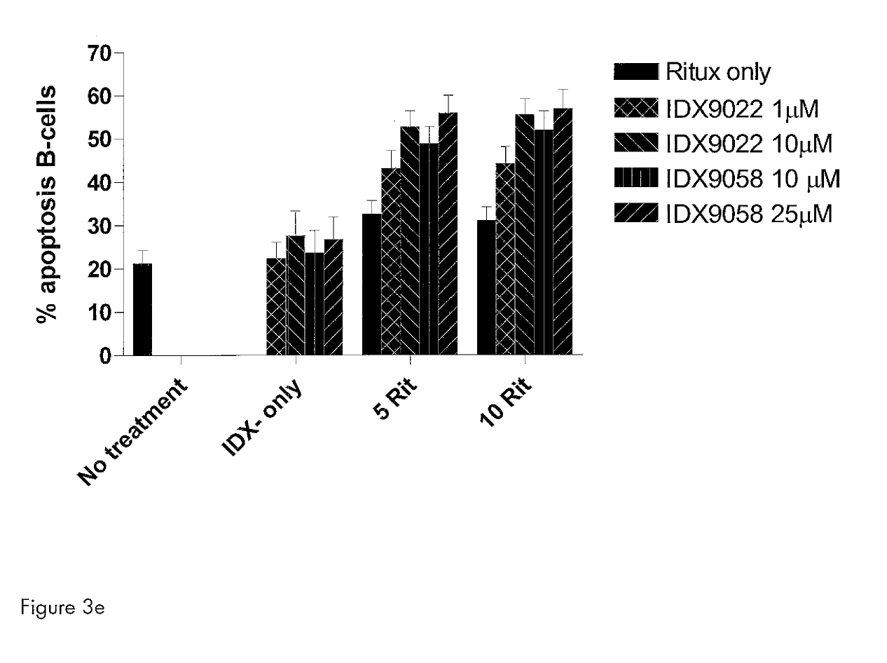

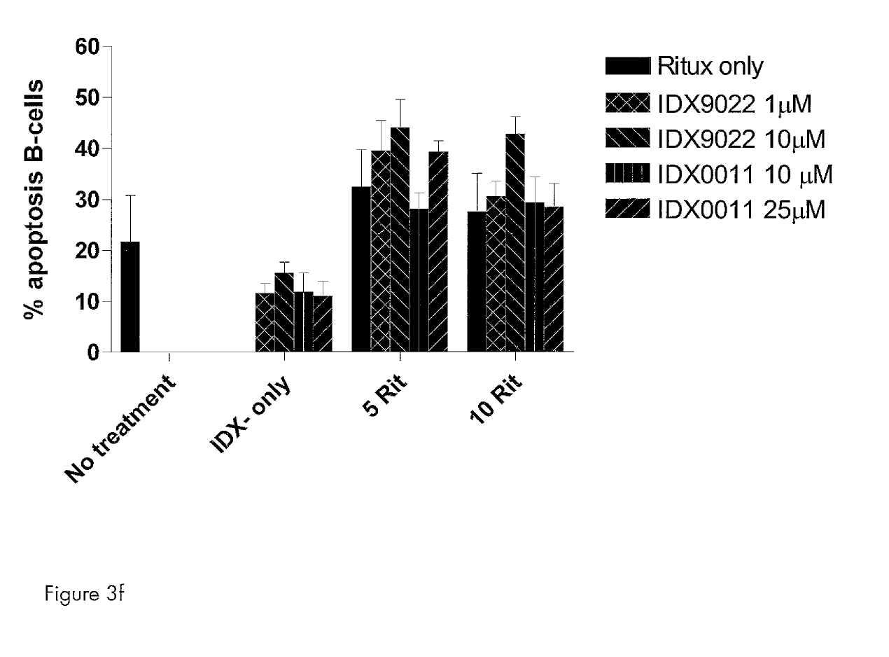

When cells were treated with rituximab after IDX-treatment, there was a marked increase in apoptosis of B-cells mediated through ADCC (FIG. 3a). There was no increase in death of B-cells mediated through CDC (data not shown). The increase in ADCC corresponded well with the observed increase in CD20 expression as well as an increase in the activation of NK-cells. The IDX-compound that induced the highest levels of CD20 was IDX9022 (SEQ ID NO. 1) at 10 μM. Cells stimulated with 1 uM of IDX9022 showed a minor increase of ADCC when treated with rituximab, whereas cells pretreated with 10 μM of IDX9022 displayed a significant increase of ADCC (FIG. 3a). IDX0150 (SEQ ID NO. 7, Table 1), IDX0505 (SEQ ID NO. 8, Table 1) and IDX9058 (SEQ ID NO. 4, Table 1), were all almost as efficient as IDX9022 in enhancing rituximab-induced ADCC (FIGS. 3c-e, respectively), while IDX0011 did not enhance ADCC (FIG. 30. NK-cells are important effector cells for ADCC to take place and the activation of these goes well in hand with the observed increase in ADCC. The enhancement of ADCC is a desired effect in the treatment of hematologic malignancies. ADCC is more efficient in eradicating tumor cells, whereas CDC is less efficient and often associated with undesired side effects for the patient.

The order of drugs is also of importance. In a reverse experiment, cells were treated with rituximab for 48 hrs and subsequently treated with IDX-compounds for another 24 hrs. This order of combination treatment did not result in enhanced ADCC (FIG. 3b). These results shows that an up-regulation of CD20 is necessary for the combination treatment to be effective. In previous clinical trials NHL patients have been treated with rituximab before treatment with CpG-ODNs and in these patients clinical response was not improved by the addition of CpG-ODNs. In order for the combination treatment to be efficient, patients should be treated with IDX-compounds 1 or 2 days before treatment with rituximab.

Treatment of CLL cells with IDX-compounds enhances the expression of CD20 on the cell surface of CD19+ B-cells as well as the activation of NK cells. Both these effects are important steps in enhancing the observed rituximab-mediated ADCC of malignant B-CLL cells.

### 2. Receptor Expression in Pbmcs Isolated from MS Patients

### Materials and Methods

PBMCs from remitting-relapsing multiple sclerosis (RRMS) patients (n=2) were obtained using BD Vacutainer® CPT™ Cell Preparation Tubes (BD Biosciences). The cells were immediately incubated at 37° C. in a volume of 500 μl of complete RPMI-medium (containing 10% FCS, 1% PenStrep, 2 mM L-glutamine, 10 mM HEPES and 1 mM Sodium Pyruvate) in 48-well plates at a conc. of 2×10⁶ cells/ml and treated with 1, 10 and 25 μM of IDX9022. A fraction of the cells were stained CD19-PE-Cy7 and CD20-APC-Cy7 for direct analysis of surface antigen expression by FACS.

After 48 hrs, 200 μl of the cells were spun down in 96-well plates, re-suspended in 100 μl of 2% FCS (in PBS) and incubated with antibodies against CD19 and CD20 for 30 min at 4° C. The cells were then washed twice in pure PBS and subsequently analyzed by FACS using a FACS Array bioanalyzer for surface antigen expression analysis.

### Results

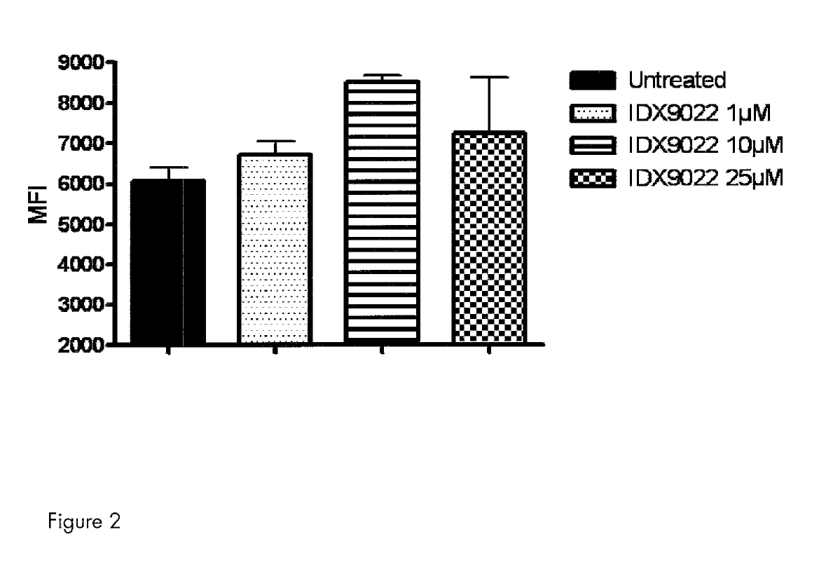

Both patients showed increased expression of CD20 upon stimulation with IDX9022 in a dose-dependent manner as shown in FIG. 2.

An increased CD20 expression was observed in PBMCs treated with the inventive compound. The inventors expect these properties of the oligonucleotide compound to be useful as alternative therapies in RRMS patients, i.e. the enhancement of antibody therapy.

### 3. Pulse Treatment of PBMCs Purified from Whole Blood from CLL Patients

### Material and Methods

IDX-compounds IDX9022 (SEQ ID NO. 1, Table 1) and IDX0150 (SEQ ID NO. 7, Table 1) were investigated for their effects on cell surface expression of CD20, CD23 and CD80 on CD19+ cells after pulsed treatment of CLL-PBMCs.

PBMCs were prepared from fresh CLL-blood and incubated in supplemented RPMI-1640 medium in 96-well plates as previously described for CLL-PBMCs. Cells were treated for 2 hrs, 6 hrs or 24 hrs with IDX9022 and IDX1050 at the following final concentrations: 0.1, 1, 10 or 25 μM. After indicated time points, cells were washed with medium twice. After the last wash the cells were resuspended in 0.2 ml medium and re-incubated at 37° C. For comparison selected wells were treated with IDX0150 or IDX9022 until the experiment was finished at 72 hrs or left untreated. After 72 hrs cells were harvested and analyzed for cell surface marker expression by FACS analysis as previously described.

### Results Pulse Experiment CD20

PBMCs were purified from whole blood from four individual CLL-patients and treated with selected IDX-compounds. Pulsed treatment of the cells resulted in an increased number of CD20 positive cells, especially at the higher concentrations of both compounds (10 and 25 μM, FIG. 4a). Overall, the longer period the cells were incubated with IDX-compounds, the more cells became CD20 positive compared to untreated cells (FIG. 4a). IDX-compound IDX9022 appear to be the most efficient of the two compounds in increasing the number of CD20 expressing B cells, since treatment of the cells for two hours, followed by wash and subsequent incubation, resulted in more CD20 positive cells than equivalent two hours treatment with IDX0150. Prolonged incubation period with 10 or 25 μM of IDX9022 induced the highest number of CD20 positive cells and corresponded with the strongest MFI-increase (data not shown). The highest numbers of CD20 positive cells obtained by IDX0150-treatment were observed when the cells were incubated with 25 μM for 72 hrs. Pulse treatment with either IDX-compound did not induce a strong increase in CD20 MFI (data not shown).

Pulsed treatment of CLL-PBMCs with IDX-compounds IDX0150 and IDX9022 resulted in increased surface expression CD23 (FIG. 4b). Overall, it seems the more IDX-compound and the longer the incubation period, the more CD23 was expressed compared to untreated cells.

IDX9022 appear to be the most efficient of the two IDX-compounds in increasing CD23, since pulsing the cells for two hours resulted in strong up-regulation of CD23, which was not observed following treatment with IDX0150 (FIG. 4b). IDX9022 also induced the highest number of CD23 positive cells compared to IDX0150. The highest increase in CD23 MFI after treatment with IDX0150 was observed after 24 hrs incubation, or when cells were harvested for flow cytometry staining after 72 hrs of treatment (FIG. 4b).

Pulsed treatment of PBMCs purified from CLL-blood with the two selected IDX-compounds resulted in increased CD80 surface expression (FIG. 4c). The longer period the cells were incubated with the IDX-compounds, the more CD80 was induced compared to untreated cells (FIG. 4c), which could be due to that a longer incubation time allows for more IDX-compounds to be taken up by the cells, and consequently more IDX-compounds will interact with TLR9.

IDX9022 appears to be the most efficient of the two IDX-compounds tested in increasing CD80, since pulsing the cells for two hours resulted in up-regulation of CD80, while two hours pulse treatment with IDX0150 did not (FIG. 4c). Long incubation periods with 10 μM of IDX9022 induced the highest percentage of CD80 positive B-cells as well as a stronger MFI-increase compared to IDX0150. The highest increase of CD80 surface expression after IDX0150-treatment was observed when cells were incubated with 25 μM of the compound throughout the experiment, i.e. for 72 hrs (FIG. 4c).

The results show that there is a time dependent induction of CD80 in response to the inventive compounds. An increased surface expression of CD80 was seen in CLL B-cells already after 2 hrs of incubation with IDX9022. The highest levels of CD80 were induced with incubation periods of 24 hrs or more.

Although the invention has been described with regard to its preferred embodiments, which constitute the best mode presently known to the inventor, it should be understood that various changes and modifications as would be obvious to one having the ordinary skill in this art may be made without departing from the scope of the invention as set forth in the claims appended hereto.

### REFERENCES

Keating M. J. Management of chronic lymphocytic leukemia: a changing field. Rev Clin Exp Hematol 2002; 6(4):350-65
Krieg A. M., Yi A K, Matson S, Waldschmidt T J, Bishop G A, Teasdale R, Koretzky G A, Klinman D M. CpG motifs in bacterial DNA trigger direct B-cell activation. Nature. 1995 April 6; 374(6522):546-9.
Krieg A. M. Therapeutic potential of Toll-like receptor 9 activation. Nat Rev Drug Discov 2006 (5) 471-484.
Pathan N I, Chu P, Hariharan K, Cheney C, Molina A, Byrd J. Mediation of apoptosis by and antitumor activity of lumiliximab in chronic lymphocytic leukemia cells and CD23+ lymphoma cell lines. Blood. 2008, February 1; 111 (3):1594-602.
Pescovitz M. D. Rituximab, an Anti-CD20 Monoclonal Antibody: History and Mechanism of Action. Am J Transpl. 2006 (6): 859-866.
Reff M. E., Garner K., Chambers K. S., Chinn P. C., Leonard J. E., Raab R., Newman R. A., Hanna N., Anderson D. R. Depletion of B-cells in vivo by a chimeric mouse human monoclonal antibody to CD20. Blood. 1994 Jan. 15; 83(2):435-45.
Strome S. E, et al. A mechanistic perspective of monoclonal antibodies in cancer therapy beyond target-related effects. Oncologist 2007, 12: 1084-95
Wooldridge J. E, et al., CpG DNA and cancer immunotherapy: orchestrating the antitumour immune response. Curr Opin Oncol. 2003 November, vol. 15, no. 6, p. 440-5.

1-23. (canceled)

24. An isolated and substantially purified oligonucleotide selected from the group consisting of SEQ ID NO: 1-6, wherein at least one nucleotide has a phosphate backbone modification, said phosphate backbone modification chosen from a phosphorothioate or a phosphorodithioate modification.

25. A pharmaceutical composition comprising an oligonucleotide according to claim 24.

26. A pharmaceutical composition comprising an oligonucleotide according to claim 24, and further comprising a pharmacologically compatible and physiologically acceptable excipient or carrier selected from the group consisting of saline, liposomes, surfactants, mucoadhesive compounds, enzyme inhibitors, bile salts, absorption enhancers, cyclodextrins, and combinations thereof.

27. A method for enhancing a treatment of a condition wherein CD20 expressing cells participate in the pathogenesis of said condition, wherein an oligonucleotide selected from the group consisting of SEQ ID NO: 1-6, or a pharmaceutical composition comprising said oligonucleotide, is administered in a dose sufficient to induce an up-regulation of the cell surface antigen CD20, and wherein said oligonucleotide is administered before said treatment.

28. A method according to claim 27, wherein the oligonucleotide is selected from the group consisting of SEQ ID NO: 1 (IDX9022) and SEQ ID NO: 4 (IDX9058).

29. A method according to claim 27, wherein the oligonucleotide is SEQ ID NO: 1 (IDX9022).

30. A method for enhancing a treatment of a condition wherein CD20 expressing cells participate in the pathogenesis of said condition, wherein an oligonucleotide selected from the group consisting of SEQ ID NO: 7 (IDX0150) and SEQ ID NO: 8 (IDX0505), or a pharmaceutical composition comprising said oligonucleotide, is administered in a dose sufficient to induce an up-regulation of the cell surface antigen CD20, and wherein said oligonucleotide is administered before said treatment.

31. A method according to claim 27, wherein said condition is cancer, and said oligonucleotide is administered in a dose effective to induce an endogenous production of at least one cytokine and an up-regulation of the expression of the cell surface antigen CD20.

32. A method according to claim 27, wherein said condition is multiple sclerosis.

33. A method according to claim 27, wherein the route of administration is selected from the group consisting of intravenous, subcutaneous, mucosal, intramuscular, and intraperitoneal administration.

34. A method according to claim 27, wherein the route of administration is mucosal administration selected from the group consisting of nasal, oral, gastric, ocular, rectal, urogenital and vaginal administration.

35. A method according to claim 27, wherein the oligonucleotide is administered in an amount of about 1 μg to about 2000 μg per kg body weight.

36. A method according to claim 27, wherein the oligonucleotide is administered in combination with an immunotherapy, and said oligonucleotide is administered in advance of said immunotherapy, preferably about 6, about 12, about 24, or about 48 hours before said therapy.

37. A method according to claim 27, wherein the oligonucleotide is administered in combination with an immunotherapy, and said oligonucleotide is administered in advance of said immunotherapy, preferably about 3 days, or about 5, 7, or 14 days before said therapy.

38. A method for increasing the therapeutic effect of a cell surface antigen targeted therapy, wherein said target is CD20, wherein an oligonucleotide selected from the group consisting of SEQ ID NO: 1-6, or a pharmaceutical composition comprising said oligonucleotide, is administered in a dose sufficient to induce an up-regulation of the cell surface antigen CD20.

39. A method according to claim 38, wherein the oligonucleotide is selected from the group consisting of SEQ ID NO: 1 (IDX9022) and SEQ ID NO: 4 (IDX9058).

40. A method according to claim 38, wherein the oligonucleotide is SEQ ID NO: 1 (IDX9022).

41. A method for increasing the therapeutic effect of a cell surface antigen targeted therapy, wherein said target is CD20, wherein an oligonucleotide selected from the group consisting of SEQ ID NO: 7 (IDX0150) and SEQ ID NO: 8 (IDX0505), or a pharmaceutical composition comprising said oligonucleotide, is administered in a dose sufficient to induce an up-regulation of the cell surface antigen CD20, and wherein said oligonucleotide is administered before said treatment.

42. A method according to claim 38, wherein said condition is cancer, and said oligonucleotide is administered in a dose effective to induce an endogenous production of at least one cytokine and an up-regulation of the expression of the cell surface antigen CD20.

43. A method according to claim 38, wherein said condition is multiple sclerosis.

44. A method according to claim 38, wherein the route of administration is selected from the group consisting of intravenous, subcutaneous, mucosal, intramuscular, and intraperitoneal administration.

45. A method according to claim 38, wherein the route of administration is mucosal administration selected from the group consisting of nasal, oral, gastric, ocular, rectal, urogenital and vaginal administration.

46. A method according to claim 38, wherein the oligonucleotide is administered in an amount of about 1 μg to about 2000 μg per kg body weight.

47. A method according to claim 38, wherein the oligonucleotide is administered in combination with an immunotherapy, and said oligonucleotide is administered in advance of said immunotherapy, preferably about 6, about 12, about 24, or about 48 hours before said therapy.

48. A method according to claim 38, wherein the oligonucleotide is administered in combination with an immunotherapy, and said oligonucleotide is administered in advance of said immunotherapy, preferably about 3 days, or about 5, 7, or 14 days before said therapy.

49. A method according to claim 38, said therapy is selected from the group consisting of an immunological therapy, treatment with antibodies, the administration of steroids, cortisone treatment, interferon treatment, or a combination of any of these.

50. A method according to claim 38, wherein said treatment includes the administration of an antibody.

51. A method of increasing the efficiency of an immunotherapy directed towards a specific antigen target, wherein the method comprises the steps of:
a) collecting a sample from said patient e.g. tissue (blood, biopsy, etc) and quantifying the expression of the antigen of interest in said sample;
b) adding one oligonucleotide selected from the group consisting of SEQ ID NO: 1-8 to said sample;
c) determining whether the expression of said antigen can be up-regulated in the sample by the addition of said oligonucleotide;
d) administering the oligonucleotide to said patient in an amount effective to up-regulate the expression of said antigen; and
e) administering an antibody drug to said patient, said antibody drug being directed to the antigen of interest.

52. The method according to claim 51, wherein said antigen of interest is CD20.

53. The method of claim 51, wherein said disease is cancer.

54. The method of claim 51, wherein said disease is multiple sclerosis.
